(19) United States
(12) Patent Application Publication
(10) Pub. No.: US 2012/0316431 A1
(43) Pub. Date: Dec. 13, 2012

(54) METHOD FOR ACQUIRING HIGH DENSITY MAPPING DATA WITH A CATHETER GUIDANCE SYSTEM

(75) Inventors: Yehoshua Shachar, Santa Monica, CA (US); Bruce Marx, Ojai, CA (US); David Johnson, West Hollywood, CA (US); Leslie Farkas, Ojai, CA (US); Steven Kim, New York, NY (US)

(73) Assignee: MAGNETECS, INC., Inglewood, CA (US)

(21) Appl. No.: 13/470,084

(22) Filed: May 11, 2012

Related U.S. Application Data
(63) Continuation of application No. 12/582,588, filed on Oct. 20, 2009, now abandoned.

(51) Int. Cl. A61M 25/095

(52) U.S. Cl. 600/424

(57) ABSTRACT

The invention is a method of rotating a catheter while it is manually guided in order to increase the volume of space it passes through during a geometric mapping procedure as to provide a higher and more uniform location data point cloud density in a volumetric mapping system.

### RELATED APPLICATIONS

This application is a continuation of U.S. patent application Ser. No. 12/582,588, filed Oct. 20, 2009, the entirety of which is incorporated herein by reference and is to be considered part of this specification.

### BACKGROUND

1. Field of the Invention

The invention relates to the systems and methods for guiding an invasive medical device within a patient for the purpose of mapping anatomical cavities.

2. Description of the Prior Art

Existing cardiac mapping software generates surface geometry from a location data point cloud of where the catheter has been. The chamber geometry is generated from this location data point cloud. The geometric surface location is based on the limits of the point cloud and data point density at those limits. If an insufficient number of points is gathered in a particular location, those few location points may be rejected as anomalous data and the surface will not be accurately generated. Prior art systems do not generate a sufficiently consistent and repeated motion through the cardiac region to generate a sufficient cloud density throughout the chamber.

### SUMMARY

The system described herein solves these and other problems by incorporating an additional motion algorithm into a catheter guidance system that rotates the catheter about the current catheter positioning vector. As the operator moves the catheter within the desired region, the catheter rotates in a controlled manner as to produce a higher density location data point cloud. This rotation is too difficult for the operator to perform manually in a consistent manner. The motion algorithm gives the operator the effective results that would be given by a catheter with more electrodes, but allows the operator to operate in smaller regions that would be inaccessible to the larger mapping catheters.

In one embodiment, the catheter is controlled by a magnetic guidance system, such as described in patent application Ser. No. 11/697,690, Shachar, et al., “METHOD AND APPARATUS FOR CONTROLLING CATHETER POSITIONING AND ORIENTATION”. The Cartesian location of each catheter electrode is continuously recorded by mapping system and these locations are sent by network data connection to the position control system for closed-loop control of catheter position. The mapping system is used to record the location data point cloud and generate the chamber geometry while the operator uses the magnetic guidance system to manipulate the catheter about the chamber. In one embodiment, the motion algorithm is manually activated by a magnetic guidance system control button, and can be turned on or off by the operator.

### BRIEF DESCRIPTION OF THE DRAWINGS

FIG. 1 is a block diagram of the placement of the motion algorithm within a catheter mapping and navigation system.

FIG. 2 is a detailed block diagram of the motion algorithm's manipulation of the catheter positioning vector.

FIG. 3 is an illustration showing the relationship between the catheter's desired position (DP) and its modified desired position (DP*).

FIG. 4 is a vector diagram depicting the time-based calculation of DP* from DP.

### DETAILED DESCRIPTION

In the field of navigating surgical tools for mapping coronary chambers or other cavities and orifices, a tool is manipulated about the chamber while a mapping system records the tool's location. These tool locations are assembled to form a location point cloud which defines the operational workspace volume. A geometric manifold representing the chamber geometry is then defined at the limits of this location point cloud. This geometry is later used by the operator as a positional reference and diagnostic tool.

The tool location is detected at each of its position detection electrodes. Some mapping catheters will have twenty or more of these electrodes, which quickly produces a very high density point cloud within the chamber. These catheters can also be very large and constructed as balloons or multiple-appendage devices. When mapping the associated vasculature of the chamber, the larger catheters either have difficulty reaching into the location or will unduly distort the tissue in an attempt to fit, so smaller catheters are often used for additional detail. These catheters have as few as four position detection electrodes and therefore, do not produce as dense of a location data point cloud for the same amount of motion. Under manually-controlled manipulation, these smaller catheters will often miss details within the vasculature or give an incomplete geometric definition of the vascular ostia.

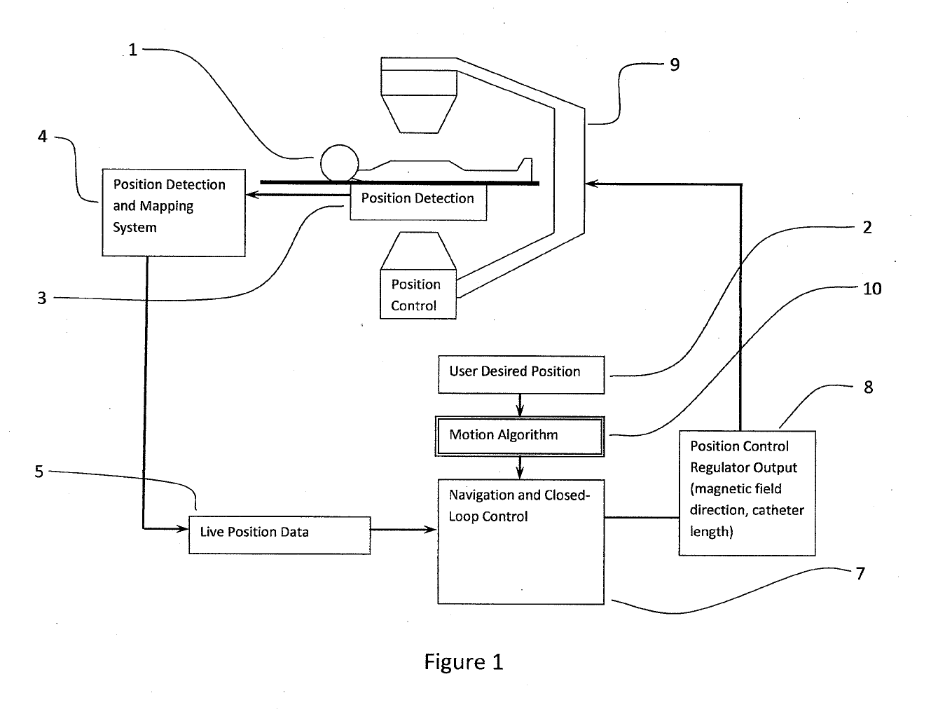

FIG. 1 is a block diagram of the placement of the motion algorithm within a catheter mapping and navigation system. The patient 1 is placed within the catheter position control system hardware 9. The catheter position detection hardware 3 is used by the position detection and mapping system 4 to send the live actual position of the catheter 5 to the navigation and closed-loop control system 7. The navigation and closed-loop control system 7 adjusts the magnetic field and catheter length values 8 and sends them to the position control hardware 9. The operator inputs the user desired position (DP) 2 for the catheter through the use of a joystick or mouse (not shown). This desired position, DP, is modified by the motion algorithm 10 before it is sent to the navigation and closed-loop position control module 7.

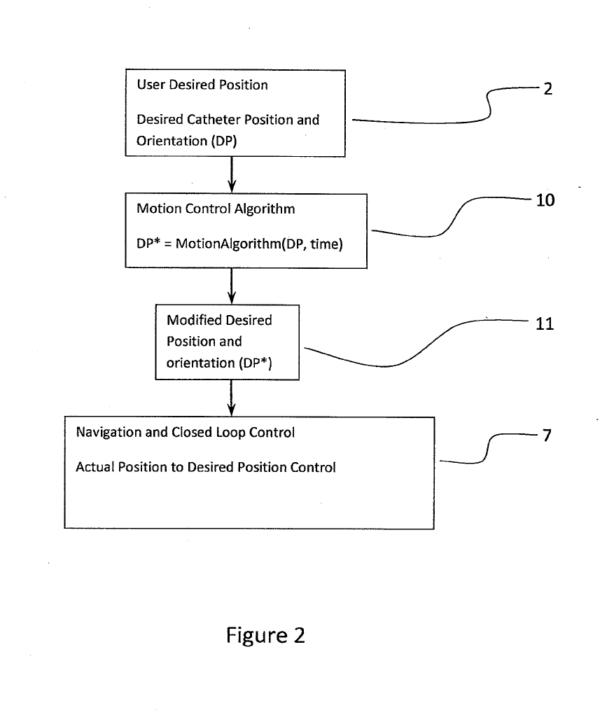

FIG. 2 is a detailed block diagram of the motion algorithm's manipulation of the catheter positioning vector. The user defined desired position, DP 2, is modified by the motion control algorithm 10 to generate the modified desired position, DP* 11. DP* is used by the navigation and closed-loop control module 7 in place of the raw user defined desired position, DP 2.

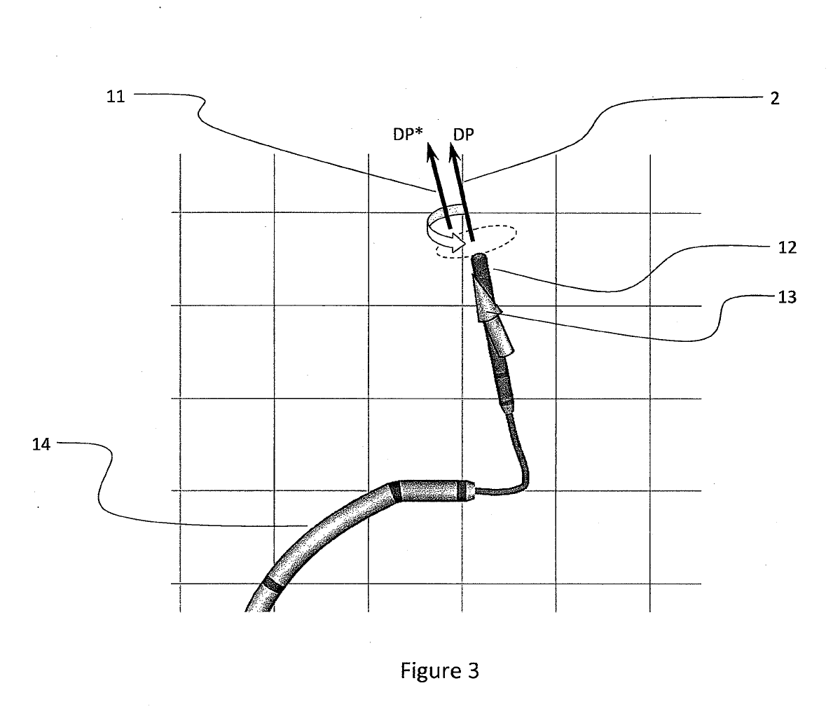

FIG. 3 is an illustration showing the relationship between the catheter's desired position (DP) and its modified desired position (DP*). The catheter 12 emerges from within the sheath 14 and is manually manipulated through the use of magnetic forces and torques. The magnetic indicator 13 indicates the actual direction of the magnetic field. The desired position, DP 2, is represented here as being identical to the actual location and direction of the catheter tip (AP), which is representative of a catheter that has been moved to its closed-loop rest position. The modified desired position, DP* 11 is a vector in the same direction as DP, but orbits at a relatively fixed distance.

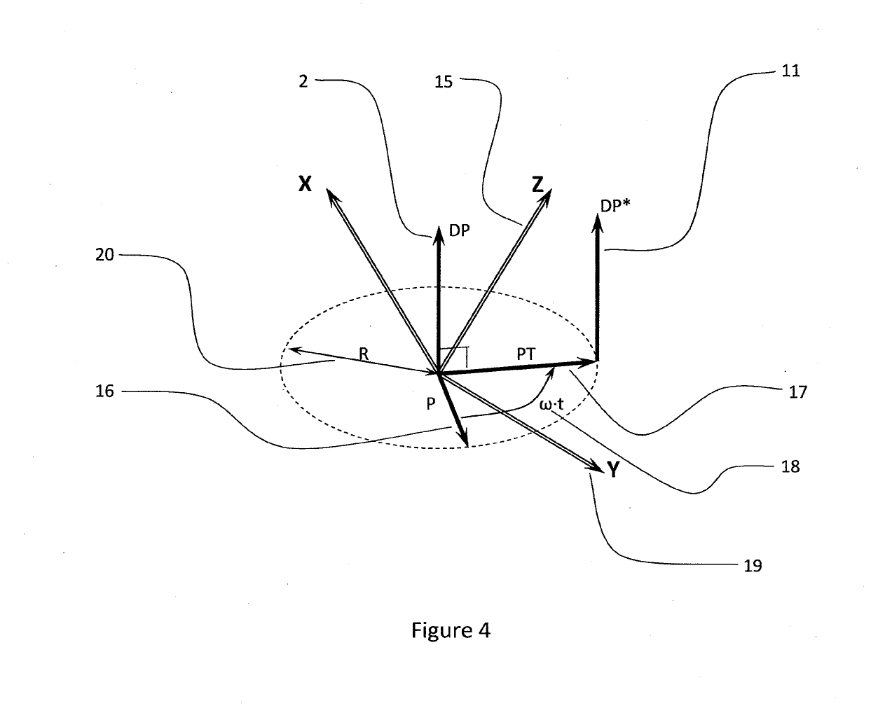

FIG. 4 is a vector diagram depicting the time-based calculation of DP* from DP. Both DP and DP* represent the six-degree-of-freedom positions and orientations of a catheter. To locate DP* 11 with respect to DP 2, the vector P 16 is calculated as the normalized cross product of the desired position DP 2 and the global coordinate Z axis 15, multiplied by the orbital radius, R 20. Where DP and Z are coincident, P 16 is set to the direction of the Y axis 19. Equation 4.1 is the derivation of the mutually perpendicular reference vector, P 16.

P=R*DP×Z/|DP×Z| 4.1

FIG. 4 further depicts the calculation of the current position offset of the DP* vector, PT 17. Using standard vector equations, the perpendicular vector P 16 is rotated about the desired position DP 2 by the angle defined by the desired angular velocity multiplied by the current time, (ω.t) 18. The result is the offset unit vector PT 17. The modified desired position DP* is the addition of the desired position DP 2 and the offset vector PT 17. The modified desired orientation component of DP* is substantially identical to that of DP.

$$\begin{matrix}PT = P rotated about DP by angle ( ω \cdot t ) . & 4.2 \\ {DP}^{*} = DP + PT & 4.3\end{matrix}$$

It is to be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but can be used alone or combined in other combinations. The excision of any disclosed element of the invention is explicitly contemplated as within the scope of the invention.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense, an equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a sub combination or variation of a sub combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the invention is limited only by the claims.

What is claimed is:

1. A system for acquisition of mapping data, comprising:
a sensor that outputs sensor data related to a position of a catheter;
a mapping system that receives said sensor data and computes a catheter actual position;
a user control device that produces a user control output related to a desired position;
a motion module that computes a plurality of modified desired positions based on said desired position; and
a closed-loop control system that receives said actual position and said plurality of modified desired positions and produces output control data that is provided to a position control system, said position control system moving a physical position of said catheter according to said output control data, said output control data configured to move said catheter to each of said modified desired positions, said mapping system configured to produce a map of a body cavity using catheter actual positions corresponding to each of said plurality of modified desired positions.

2. The system of claim 1, wherein said plurality of modified desired positions correspond to positions about said desired position.

3. The system of claim 1, wherein said plurality of modified desired positions correspond to positions arranged approximately in a circle about said desired position.

4. The system of claim 1, wherein said plurality of modified desired positions correspond to positions arranged in an orbit about said desired position.
